(19) United States
(12) Patent Application Publication
(10) Pub. No.: US 2007/0004911 A1
(43) Pub. Date: Jan. 4, 2007

(54) Silylated oligonucleotide compounds

(76) Inventors: David John Moody, STIRLINGSHIRE (GB); Paul McCormac, Stirlingshire (GB); Sarah Anne Barron, Stirlingshire (GB)

Correspondence Address: MORGAN LEWIS & BOCKIUS LLP, 1111 PENNSYLVANIA AVENUE NW, WASHINGTON, DC, 20004, US

(21) Appl. No.: 10/550,217

(22) PCT Filed: Mar. 19, 2004
(86) PCT No.: PCT/GB04/01196

§ 371 Date: Aug. 14, 2006

(30) Foreign Application Priority Data
Mar. 24, 2003 (GB) 0306657.8

(51) Int. Cl. C07H 21/02; C07H 21/04

(52) U.S. Cl. 536/23.1; 536/25.33

(57) ABSTRACT

Oligonucleotide comprising at least one internucleotide phosphorus atom protected with a group of formula —X^{a}SiR³R⁴R⁵ provided. X^{a} represent 0 or S, and R³, R⁴ and R⁵ each independently are optionally substituted hydrocarbyl groups, selected such that that total number of carbon atoms in R³ plus R⁴ plus R⁵ is 4 or more. Process for the preparation of these oligonucleotides, intermediate compounds useful therein, and process for the preparation of the intermediate compounds are also provided.

The present invention concerns a method for the synthesis of oligonucleotides, silylated oligonucleotide derivatives, intermediate compounds, reagents, and methods for the preparation thereof.

Oligonucleotides substituted with trimethylsilyloxy moieties on the phosphorus backbone have been proposed by a number of researchers. See for example Brill, Tetrahedron Letters Vol 36, No. 5, pp 703-706 (1995); Fuji et al, Tetrahedron, Vol 43, No. 15, pp 3395-3407 (1987); Kume et al, J. Org. Chem. 1984, 49, pp 2139-2143; Seela et al, J. Chem. Soc. Chem. Commun. 1990, p1154-1159; and Seela et al, J. Org. Chem. Vol. 56, No. 12. pp 3861-3869 (1991). However, when such compounds are oxidised or sulphurised, the trimethylsilyl group is displaced. The presence of bulky organosilyl groups may offer advantages in the purification of the oligonucleotide. Accordingly, it would be desirable to identify silylated oligonucleotides in which the silyl group is not displaced during oxidation or sulphurisation.

According to one aspect of the present invention there is provided an oligonucleotide comprising at least one internucleotide phosphorus atom protected with a group of formula —X^{a}SiR³R⁴R⁵ wherein X^{a} represents O or S, preferably O, and R³, R⁴ and R⁵ each independently are optionally substituted hydrocarbyl groups, selected such that that total number of carbon atoms in R³ plus R⁴ plus R⁵ is 4 or more. In certain embodiments, a single group of formula —X^{a}SiR³R⁴R⁵ is present located at the terminal internucleotide linkage, preferably at the 5′-end. In certain other embodiments, commonly at least 50%, more preferably at least 75% and most preferably 100% of the internucleotide phosphorus atoms are protected with a group of formula —SiR³R⁴R⁵.

A particular embodiment of the present invention provides compounds of Formula (1): 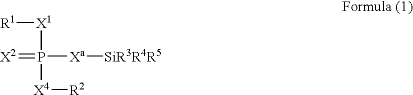
wherein:
R¹ and R² independently are nucleoside, nucleotide or oligonucleotide moieties;
R³, R⁴ and R⁵ each independently are optionally substituted hydrocarbyl groups, selected such that that total number of carbon atoms in R³ plus R⁴ plus R⁵ is 4 or more;
X^{a} represents O or S, preferably O;
X¹ and X⁴ are each independently —O—, —S—, —CH₂— or NRⁿ, where Rⁿ represents H or
C₁₋₄ alkyl, preferably both of X¹ and X⁴ being O; and
X² is O or S, and preferably S.

Nucleoside, nucleotide or oligonucleotide moieties that can be represented by R¹ and R² include deoxyribonucleosides, deoxyribonucleotides, oligodeoxyribonucleotides, ribonucleosides, ribonucleotides, oligoribonucleotides, and oligonucleotides comprising mixtures of deoxyribo- and ribonucleosides and nucleotides. The nucleosides, nucleotides or oligonucleotides may be modified by one or modifications known in the field of oligonucleotide chemistry, for example ribonucleosides, ribonucleotides or oligoribonucleotides may be modified at one or more of the 2′-positions by the presence of a 2′-alkoxy group, such as a methoxy or methoxyethoxy group. Deoxyribonucleosides, deoxyribonucleotides or oligodeoxyribonucleotides may be modified at the 2′-position by the presence of a substituent, such as a halo group, especially a fluoro group, or by an alkenyl group such as an allyl group. A basic nucleoside or nucleotide moieties may also be present. In many embodiments, the nucleosides, nucleotide or oligonucleotides represented by R¹ and R² will represent the natural D-isomer. However, either or both of R¹ and R² may represent an unnatural isomer, for example an L-isomer or a B-anomer, either in whole or in part. One or both of R¹ and R² may comprise one or more protecting groups. Examples of such protecting groups, and the positions which they can be employed to protect, are well known to those skilled in the art, and include trityl, monomethoxytrityl and dimethoxytrityl groups, levulinoyl groups, isobutyryl groups, benzoyl groups, acetyl groups and carbonate groups, such as BOC and especially FMOC. When either of R¹ and R² represents an oligonucleotide, one or more of the internucleotide linkages therein may be protected by a group of formula —X^{a}SiR³R⁴R⁵.

In many embodiments, X¹ connects the 3′-position of a ribose or deoxyribose moiety of R¹ to the phosphorus, P. However, it will be recognised that X¹ may connect the 5′-position of a ribose or deoxyribose moiety of R¹ to the phosphorus, P.

In many embodiments, X⁴ connects the 5′-position of a ribose or deoxyribose moiety of R² to the phosphorus, P. However, it will be recognised that X⁴ may connect the 3′-position of a ribose or deoxyribose moiety of R² to the phosphorus, P.

Either of R¹ and R² may be attached to a solid support, commonly via a cleavable linker. In many embodiments, R² is attached to a solid support via a cleavable linker, preferably via the 3′-position of a ribose or deoxyribose moiety. Examples of cleavable linkers include base labile linkers such as succinyl linkers, and acid labile linkers such as trityl linkers.

Hydrocarbyl groups which can be represented by one or more of R³, R⁴ and R⁵ include any optionally substituted hydrocarbyl groups that allow the P(III) centre to react with a sulphurising agent or oxidation agent, especially optionally substituted alkyl groups, optionally substituted aryl groups and mixtures thereof, such as aralkyl, especially benzyl, groups.

When at least one of R³, R⁴ and R⁵ represents an optionally substituted alkyl group, it is preferably an optionally substituted C₁₋₁₂ alkyl, more preferably an optionally substituted C₁₋₈alkyl and particularly an optionally substituted C₁₋₄alkyl group.

When at least one of R³, R⁴ and R⁵ represents an optionally substituted aryl group, it is preferably an optionally substituted phenyl group.

R³, R⁴ and R⁵ may be the same or different.

It is particularly preferred that each of R³, R⁴ and R⁵ is selected from the group consisting of methyl, ethyl, propyl and butyl groups. In many embodiments, at least one of represents a branched alkyl group, such as an isopropyl, isobutyl, and especially a tert-butyl, group.

Preferably the total number of carbon atoms in R³, R⁴ and R⁵ is 5 or greater, and particularly from 6 to 10.

In certain embodiments, one of R³, R⁴ and R⁵ is ethyl or propyl, especially isopropyl, and the other two are methyl, and in certain other embodiments, one of R³, R⁴ and R⁵ is tert-butyl and the other two are methyl.

Optional substituents for R³, R⁴ and R⁵ are preferably selected from the group consisting of alkyl (preferably C₁₋₄-alkyl), optionally substituted alkoxy (preferably C₁₋₄-alkoxy), optionally substituted aryl (preferably phenyl), optionally substituted aryloxy (preferably phenoxy), polyalkylene oxide (preferably polyethylene oxide or polypropylene oxide), carboxy, phosphato, sulpho, nitro, cyano, halo, ureido, —SO₂F, hydroxy, ester, —NR^{a}R^{b}, —COR^{e}, —CONR^{a}R^{b}, —NHCOR^{a}, carboxyester, sulphone, and —SO₂NR^{a}R^{b} wherein R^{a} and R^{b} are each independently H or optionally substituted alkyl (especially C₁₋₄alkyl) or, in the case of —CONR^{a}R^{b} and —SO₂NR^{a}R^{b}, R^{a} and R^{b} together with the nitrogen atom to which they are attached represent an aliphatic or aromatic ring system; or a combination thereof.

Preferred compounds of Formula (1) include compounds of Formula (2):

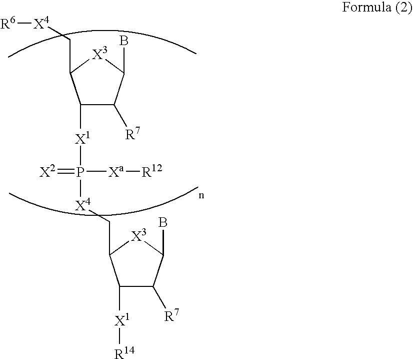

In compounds of Formula (2), X^{a} for each occurrence is independently —O— or —S—. Preferably X^{a} is 0 at each occurrence. X¹ and X⁴ are, independently, —O—, —CH₂—, —S— or NRⁿ, where Rⁿ represents H or C₁₋₄ alkyl. Preferably, X¹ and X⁴ are —O— at every occurrence. X² for each occurrence is O or S, preferably S. X³ for each occurrence is, independently, —O—, —S—, —CH₂—, or —(CH₂)₂—. Preferably, X³ is —O— at every occurrence. In a more preferred embodiment, X¹ and X³ are all —O— at every occurrence. R⁶ is H, an alcohol protecting group, an amino protecting group or a thio protecting group. Preferably, R⁶ is a protecting group which is removable under conditions orthogonal to a group of formula X^{a}—SiR³R⁴R⁵. R⁷ for each occurrence is, independently, —H, —F—OR⁸, —NR⁹R¹⁰, —SR¹¹, or a substituted or unsubstituted aliphatic group, such as methyl or allyl. R¹² for each occurrence is, independently, a phosphorus protecting group, such as a group of formula —CH₂CH₂CN, a substituted or unsubstituted aliphatic group, —R¹³, —CH₂CH₂—Si(CH₃)₂C₆H₅, —CH₂CH₂—S(O)₂—CH₂CH₃ or —CH₂CH₂—C₆H₄—NO₂, provided that at least one R¹² represents a group of formula —SiR³R⁴R⁵, in which R³, R⁴ and R⁵ are as previously defined. In certain embodiments, each R¹² represents a group of formula —SiR³R⁴R⁵. In certain other embodiments, only one R¹² represents a group of formula —SiR³R⁴R⁵, advantageously being located at the 5′-terminal internucleotide phosphorus. R⁸ for each occurrence is, independently, —H, a substituted or unsubstituted aliphatic group (e.g., methyl, ethyl, methoxyethyl or allyl), a substituted or unsubstituted aryl group, a substituted or unsubstituted aralkyl, an alcohol protecting group, or —(CH₂)_{q}—NR^{x}R^{y}. R⁹ and R¹⁰ for each occurrence are each, independently, —H, a substituted or unsubstituted aliphatic group, or an amine protecting group. Alternatively, R⁹ and R¹⁰ taken together with the nitrogen to which they are attached are a heterocyclyl group. R¹¹ for each occurrence is, independently, —H, a substituted or unsubstituted aliphatic group, or a thio protecting group. R¹³ is for each occurrence is, independently, a substituted or unsubstituted aliphatic group, a substituted or unsubstituted aryl group or a substituted or unsubstituted aralkyl group. R^{x} and R^{y} are each, independently, —H, a substituted or unsubstituted aryl group, a substituted or unsubstituted heteroaryl group, a substituted or unsubstituted aliphatic group, a substituted or unsubstituted aralkyl group, a substituted or unsubstituted heteroaralkyl group or an amine protecting group. Alternatively, R^{x} and R^{y} taken together with the nitrogen to which they are attached form a heterocyclyl group. q is an integer from 1 to about 6. B is —H, a natural or unnatural nucleobase, or a protected natural or unnatural nucleobase. R¹⁴ is H a hydroxy protecting group, a thio protecting group, an amino protecting group, —(CH₂)_{q}—NR^{x}R^{y}, a solid support, or a cleavable linker attached to a solid support, such as a group of the formula —Y-L-Y—R¹⁵. Y for each occurrence is, independently, a single bond, —C(O)—, —C(O)NR¹⁸—, —C(O)O—, —NR¹⁶— or —O—. L is a linker which is preferably a substituted or unsubstituted aliphatic group or a substituted or unsubstituted aromatic group, for example a trityl group. More preferably, L is an ethylene group. R¹⁵ is —H, a substituted or unsubstituted aliphatic group or a substituted or unsubstituted aromatic group. R¹⁵ is any solid support suitable for solid phase oligonucleotide synthesis known to those skilled in the art. Examples of suitable solid supports include controlled-pore glass, polystyrene, microporous polyamide, such as poly(dimethylacrylamide), and polystyrene coated with polyethylene. In many embodiments, R¹⁴ represents a cleavable linker, such as a succinyl, oxaloyl or trityl linker, attached to a solid support. n is a positive integer, preferably from 1 to 100, for example up to 75, commonly up to 50, and particularly from 8 to 40.

Natural and unnatural nucleobases that can be represented by B include adenine, guanine, cytosine, thymine, and uracil and modified bases such as 7-deazaguanine, 7-deaza-8-azaguanine, 5-propynylcytosine, 5-propynyluracil, 7-deazaadenine, 7-deaza-8-azaadenine, 7-deaza-6-oxopurine, 6-oxopurine, 3-deazaadenosine, 2-oxo-5-methylpyrimidine, 2-oxo-4-methylthio-5-methylpyrimidine, 2-thiocarbonyl-4-oxo-5-methylpyrimidine, 4-oxo-5-methylpyrimidine, 2-amino-purine, 5-fluorouracil, 2,6-diaminopurine, 8-aminopurine, 4-triazolo-5-methylthymine, 4-triazolo-5-methyluracil and hypoxanthine.

According to a second aspect of the present invention, there is provided a process for the preparation of a compound of Formula (1) as defined above, which comprises oxidising or sulfurising a compound of Formula (3):

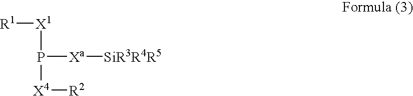

wherein R¹, R², R³, R⁴, R⁵, X^{a}, X¹ and X⁴ are as defined above.

Compounds of Formula (3) form another aspect of the present invention.

The sulfurisation agent employed in the process according to the second aspect of the present invention is any agent able to add sulfur to compounds of Formula (3), such as elemental sulfur.

Preferably the sulfurisation agent is an organic sulfurisation agent.

Examples of organic sulfurisation agents include 3H-benzodithiol-3-one 1,1-dioxide (also called “Beaucage reagent”), dibenzoyl tetrasulfide, phenylacetyl disulfide, N,N,N′,N′-tetraethylthiurarn disulfide, and 3-amino-[1,2,4]dithiazole-5-thione (see U.S. Pat. No. 6,096,881, the entire teachings of which are incorporated herein by reference).

Typical reaction conditions for sulfurisation of an oligonucleotide using the above agents can be found in Beaucage, et al., Tetrahedron (1993), 49, 6123, which is incorporated herein by reference.

Preferred sulfurisation reagents are 3-amino-[1,2,4]dithiazole-5-thione and phenylacetyl disulfide.

Sulfurisation of an oligonucleotide may be carried out by, for example use of a solution of 3-amino-[1,2,4]dithiazole-5-thione in an organic solvent, such pyridine/acetonitrile (1:9) mixture or pyridine, having a concentration of about 0.05 M to about 0.2 M.

The oxidising agent employed in the process according to the second aspect of the present invention is any agent able to add oxygen to compounds of Formula (3). Examples of oxidising agents include iodine and peroxides, such as t-butylhydroperoxide

Compounds of Formulae (1), (2) and (3) may be prepared by the use of phosphoramidite chemistry, employing silyl phosphoramidites. Accordingly, a third aspect of the present invention comprises compounds of Formula (4):
R¹—X¹—P(NR¹⁷R¹⁸)—X^{a}—SiR³R⁴R⁵

wherein R¹, R³, R⁴, R⁵, X^{a} and X¹ are as previously defined, and R¹⁷ and R¹⁸ are each, independently, a substituted or unsubstituted aliphatic group, such as a C₁₋₄ alkyl group, especially an isopropyl group; a substituted or unsubstituted aryl group; or a substituted or unsubstituted aralkyl group. Alternatively, R¹⁷ and R¹⁸ taken together with the nitrogen to which they are bound form a heterocyclyl group.

Preferred compounds of the third aspect of the present invention are compounds of Formula (5):

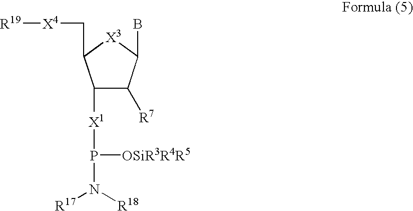

wherein R³, R⁴, R⁵, R⁷, R¹⁷, R¹⁸, B, X¹, X³ and X⁴ are as previously defined, and R¹⁹ represents an alcohol, thiol or amino protecting group, preferably a protecting group removable under conditions orthogonal to the OSiR³R⁴R⁵ group. In many embodiments, it is preferred that R¹⁷ and R¹⁸ are each alkyl groups, preferably C₁₋₄ alkyl groups, and especially isopropyl groups.

Preferred compounds of Formula (5) are compounds of Formula (6):

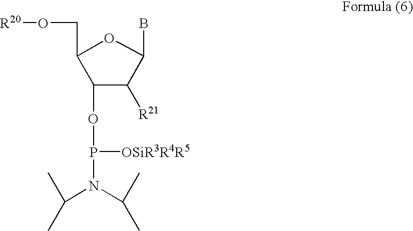

wherein R³, R⁴, R⁵ and B are as previously defined, R²⁰ represents a protecting group, preferably a protecting group removable under conditions orthogonal to the group of formula O—SiR³R⁴R⁵, such as a carbonate protecting group, especially t-butoxycarbonyl (BOC) or fluorenylmethoxycarbonyl (FMOC), and R²¹ represents H, OMe, OCH₂CH₂OCH₃, or OR²², and R²² represents a protecting group, known in the art for the protection of the 2′-hydroxy of ribonucleosides, and preferably a silyl, particularly a trialkylsilyl, and especially a tert-butyidimethylsilyl group. In particularly preferred compounds of Formula (6), R³ and R⁴ represent methyl groups, and R⁵ represents a tert-butyl group. In certain embodiments, especially where a compound of Formula (6) is employed to add the final nucleoside of a given oligonucleotide sequence, R²⁰ may represent a silyl protecting group, particularly a trialkylsilyl, and especially a tert-butyidimethylsilyl group.

Compounds of Formula (4) wherein X^{a} is O can be prepared by a) reaction between a compound of formula R¹—X¹—H, wherein R¹ and X¹ are as previously defined, and a compound of formula Z-P(NR¹⁷R¹⁸)₂ wherein R¹⁷ and R¹⁸ are as previously defined and Z represents a leaving group, preferably a chlorine atom, to form a compound of formula R¹—X¹—P(NR¹⁷R¹⁸)₂; b) hydrolysing the compound of formula R¹—X¹—P(NR¹⁷R¹⁸)₂ to form a compound of formula R¹—X¹—PH(═O)(NR¹⁷R¹⁸), the hydrolysis preferably taking place in the presence of a weak acid, such as tetrazole, S-ethyltetrazole, or an imidazole salt; and c) reacting the compound of formula R¹—X¹—PH(═O)(NR¹⁷R¹⁸) with a silylating agent of formula Y¹—SiR³R⁴R⁵ wherein Y¹ is a leaving group to form the compound of Formula (4). Examples of leaving groups which can be represented by Y include halogen, especially Cl and Br. Further examples of leaving groups include the residues from bis silylating agents, such as compounds of the formulae: 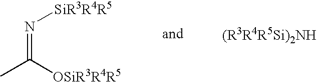
wherein R³, R⁴ and R⁵ are as previously defined.

Compounds of Formula (4) can also be prepared by reaction between a compound of formula R¹—X¹—H, wherein R¹ and X¹ are as previously defined, and a compound of formula R³R⁴R⁵Si—X^{a}—P(NR¹⁷R¹⁸)₂ wherein X^{a}, R³, R⁴, R⁵, R¹⁷ and R¹⁸ are as previously defined. The compound of formula R³R⁴R⁵Si—X^{a}—P(NR¹⁷R¹⁸)₂ can be prepared by reaction between a compound of formula Z-P(NR¹⁷R¹⁸)₂, where Z is as previously defined, and a compound of formula H—X^{a}—SiR³R⁴R⁵, preferably in the presence of a base, especially a trialkylamine. Compounds of formula R³R⁴R⁵Si—O—P(NR¹⁷R¹⁸)₂ may also be prepared by hydrolysis of a compound of formula Z-P(NR¹⁷R¹⁸)₂, to form a compound of formula H—O—P(NR¹⁷R¹⁸)₂, which is then reacted with a compound of formula Y¹—SiR³R⁴R⁵ wherein Y¹ is as described above.

According to a fourth aspect of the present invention, there is provided a process for the preparation of a compound of Formula (1) which comprises a) coupling a compound of Formula (4) as defined above with a nucleoside, nucleotide or oligonucleotide, comprising a free hydroxy, thiol, amino or imino group, of formula R²—OH, R²—SH or R²—NR⁶H, wherein R² and R⁶ are as previously defined, and preferably a nucleoside, nucleotide or oligonucleotide comprising a free 5′-hydroxy group, in the presence of an activator, and b) oxidising or sulfurising the product of step a). In one embodiment, the process of the fourth aspect of the present invention comprises the coupling of a compound of Formula (4) as defined above to add the final nucleotide in an oligonucleotide, the remaining nucleotides of which having been added using phosphoramidites comprising conventional phosphorus protecting groups, such as betacyanoethyloxy phosphoramidites.

Preferably the nucleoside, nucleotide or oligonucleotide comprising the free hydroxyl or thiol group is attached to a solid support, most preferably via a cleavable linker, preferably a trityl or succinyl linker. It is particularly preferred that the attachment to the solid support is via the 3′-position of a ribose or deoxyribose moiety.

A preferred embodiment of the present invention comprises a sequence of processes of the fourth aspect wherein a protected compound of Formula (4) is coupled, in the presence of an activator, to a free hydroxy group to form a protected nascent oligonucleotide, a protecting group, most preferably a 5′-protecting group, is removed from the nascent oligonucleotide to form a free hydroxy group, which is then coupled with another compound of Formula (4) in the presence of an activator. The cycle can be repeated as often as desired until the desired oligonucleotide sequence has been assembled.

The compound of Formula (4) is advantageously employed as a solution in an inert solvent. Examples of such solvents suitable for use in phosphoramidite chemistry are well known in the art, and include in particular acetonitrile, dichloromethane, THF and pyridine.

Activators for phosphoramidites which can be employed in the process of the present invention are well known in the field of oligonucleotide synthesis. Examples include tetrazole; S-ethyl tetrazole; pyridinium salts, imidazolinium salts and benzimidazolinium salts as disclosed in PCT application WO 99/62922 (incorporated herein by reference) and salt complexes formed between saccharin and organic amines, especially N-methylimidazole, pyridine and 3-methylpyridine.

A fifth aspect of the present invention provides a process for the synthesis of an oligonucleotide comprising at least one internucleotide phosphorus atom protected with a group of formula —X^{a}SiR³R⁴R⁵, wherein X^{a} represents O or S, and R³, R⁴ and R⁵ each independently are optionally substituted hydrocarbyl groups, selected such that that total number of carbon atoms in R³ plus R⁴ plus R⁵ is 4 or more which comprises reacting a silylating agent of formula Y¹—SiR³R⁴R⁵ as described above with an oligonucleotide H-phosphonate diester.

Particularly preferred trihydrocarbylsilyl donors are ethyldimethylsilyl chloride and tert-butyldimethylsilyl chloride, and especially bis(ethyldimethylsilyl)acetamide, bis(tert-butyidimethylsilyl)acetamide, bis(ethyldimethyl)disilazane and bis(tert-butyidimethyl)disilazane.

Preferred oligonucleotide H-phosphonate diesters are compounds of Formula (7):

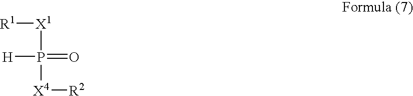

wherein R¹, R², X¹ and X⁴ are as previously defined. Most preferably, X¹ and X⁴ represent —O—.

Oligonucleotide H-phosphonate diesters can be prepared by methods well known in the art, for example by reaction between a nucleoside or oligonucleotide H-phosphonate monoester, and a nucleoside or oligonucleotide comprising a free hydroxyl or thiol group.

A preferred embodiment of the present invention comprises a sequence of processes of the fourth aspect wherein a protected nucleoside or nucleotide H-phosphonate monoesters are sequentially coupled, in the presence of an activator, to a free hydroxy group to form a protected nascent oligonucleotide, a protecting group, most preferably a 5′-protecting group, is removed from the nascent oligonucleotide to form a free hydroxy group, which is then coupled with another nucleoside or nucleotide H-phosphonate monoester in the presence of an activator. The cycle can be repeated as often as desired until the desired oligonucleotide sequence has been assembled.

In one embodiment, the process of the fifth aspect of the present invention is employed to introduce a group of formula X^{a}—Si—R³R⁴R⁵ into the terminal internucleotide linkage of a desired oligonucleotide sequence.

Activators for H-phosphonates which can be employed are those well know in the art for the formation of H-phosphonate diesters, such as diphenyl phosphorochloridate and pivaloyl chloride.

The processes according to the present invention are preferably employed to produce oligonucleotides comprising at least one internucleotide phosphorus atom protected with a group of formula —X²SiR³R⁴R⁵ as defined above, which comprise 3 or more bases. Preferably the oligonucleotide comprises 5 to 75, more preferably from 8 to 50 and particularly from 10 to 30 internucleoside linkages. Commonly, the processes of the present invention are employed to prepare compounds wherein at least 50% of the internucleoside linkages are phosphorothioated, preferably at least 75%, and most preferably 90 to 100% of the internucleoside linkages phosphorothioated.

When the processes according to the present invention are used to produce oligonucleotides then the conditions used are any of those known in the art.

Solvents which may be employed in the processes of the present invention include: haloalkanes, particularly dichloromethane; esters, particularly alkyl esters such as ethyl acetate, and methyl or ethyl propionate; nitriles, such as acetonitrile; amides, such as dimethylformamide and N-methylpyrollidinone; and basic, nucleophilic solvents such as pyridine. Preferred solvents are pyridine, dichloromethane, dimethylformamide, N-methylpyrollidinone and mixtures thereof. A particularly preferred solvent is pyridine. Organic solvents employed in the process of the present invention are preferably substantially anhydrous.

Supports for the solid phase synthesis of oligonucleotides are well known in the art. Examples include silica, controlled pore glass, polystyrene, copolymers comprising polystyrene such as polystyrene-poly(ethylene glycol) copolymers and polymers such as polyvinylacetate. Additionally, poly(acrylamide) supports, especially microporous or soft gel supports, such as those more commonly employed for the solid phase synthesis of peptides may be employed if desired. Preferred poly(acrylamide) supports are amine-functionalised supports, especially those derived from supports prepared by copolymerisation of acryloyl-sarcosine methyl ester, N,N-dimethylacrylamide and bis-acryloylethylenediamine, such as the commercially available (Polymer Laboratories) support sold under the catalogue name PL-DMA. The procedure for preparation of the supports has been described by Atherton, E.; Sheppard, R. C.; in Solid Phase Synthesis: A Practical Approach, Publ., IRL Press at Oxford University Press (1984) which is incorporated herein by reference. The functional group on such supports is a methyl ester and this is initially converted to a primary amine functionality by reaction with an alkyl diamine, such as ethylene diamine.

The processes for the synthesis of a trihydrocarbyl silyl phosphate or phosphorothioate triester in the solid state may be carried out by stirring a slurry of the substrate bonded to the solid and comprising silyl phosphite linkages in a solution of oxidising or sulfurisation agent. Alternatively, the solid support can be packed into a column, and solutions of the oxidising or sulfurisation agent can be passed through the column.

On completion of the assembly of the desired product, the product may be cleaved from the solid support, using cleavage methods appropriate for the linker, preferably following deprotection of the product.

The product of the process can be purified using one or more standard techniques known in the art, such as, ion-exchange chromatography, reverse phase chromatography, precipitation from an appropriate solvent and ultra-filtration.

Many of the compounds used herein may exist in the form of a salt. These salts are included within the scope of the present inventions.

The compounds described herein may exist in tautomeric forms other than those shown in this specification. These tautomers are also included within the scope of the present inventions.

According to a sixth aspect of the present invention, there is provided a process for the preparation of a deprotected oligonucleotide which comprises a) assembling an oligonucleotide compound comprising at least one internucleotide phosphorus atom protected with a group of formula —X^{a}SiR³R⁴R⁵ wherein X^{a}, R³, R⁴ and R⁵ are as described herein, and b) removing the SiR³R⁴R⁵ groups. The oligonucleotide compound comprising at least one internucleotide phosphorus atom protected with a group of formula —X^{a}SiR³R⁴R⁵ is advantageously prepared by a process according to the fourth or fifth aspects of the present invention. The SiR³R⁴R⁵ groups can be removed by methods known in the art for the removal of organosilyl protecting groups, for example by treatment with a source of fluoride, such as ammonium fluoride, under basic, nucleophilic conditions; by treatment with tert-butyl ammonium fluoride; or by treatment with an alkylamine-HF complex such as (C₂H₅)₃N.3HF. The SiR³R⁴R⁵ groups can be removed either before or after other protecting groups are removed. It will be recognised that this, together with the nature of the other protecting groups, may influence the choice of conditions employed. For example, the SiR³R⁴R⁵ groups may be removed by treatment with acetic acid, which treatment will also remove trityl-type protecting groups. When the oligonucleotide has been prepared whilst supported on a solid support, the SiR³R⁴R⁵ groups are commonly removed after cleavage of the oligonucleotide from the support.

The invention will now be illustrated without limitation by the following examples.

Liquid Chromatography Analysis

In the examples analysis by liquid chromatography used the following protocol:

All samples were prepared in acetonitrile;

The chromatography medium was Genesis C18, 120A, 4μ;

The dimensions of the column were 25×0.46 cm;

The flow rate was 1.0 ml/minutes;

The detector was set at 270 nm;

The run time was 30 minutes;

The elution system used the following solvents:

0 minutes=80% 0.1% aqueous ammonium acetate buffer: 20% acetonitrile

20 minutes=100% acetonitrile

22 minutes=100% acetonitrile

30 minutes=80% 0.1% aqueous ammonium acetate buffer: 20% acetonitrile.

In the examples the following abbreviations are used:

BMTBSA N,N-Bis(tert-butyldimethylsilyl)acetamide

DCM Dichloromethane

DMF N,N-Dimethylformamide

DMT 4,4′-Dimethoxytrityl

PADS Diphenyldithiocarbamate

TBDMSCI tert-Butyldimethylsilyl chloride

TEAP Triethylamine phosphate

THF Tetrahydrofuran

### EXAMPLE 1

Stage 1

### Preparation of 3M aqueous triethylamine phosphate (TEAP)

Triethylamine (410 ml) and water (400 ml) were charged to a beaker and cooled to 0-5° C. Phosphoric acid (180 g) was added slowly to the stirred mixture until the pH was in the range of pH 7 to 7.5 was reached. The solution was then transferred to a 1 L volumetric flask and diluted to 1 L with water. Prior to use TEAP was diluted with water as required.

Stage 2

### Preparation of N⁴-benzoyl-5′-O-(4,4′-dimethoxytrityl)-2′-deoxy-3′-(hydrogen phosphate)cytidine triethylammonium salt (DMT-Bz-C-H-Phos)

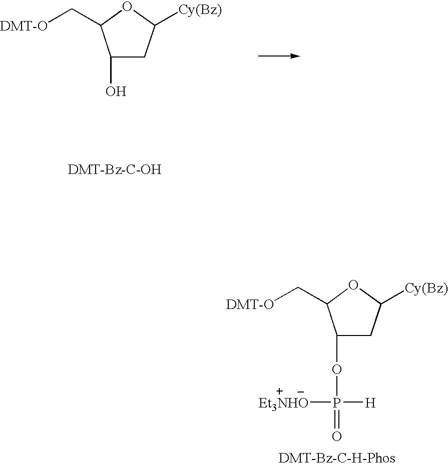

THF (416 ml) and 1H,1,2,4-triazole (16.1 g) were charged to a 1 L round-bottomed flask fitted with a thermometer, condenser, nitrogen inlet and overhead stirrer. The solution was cooled, with stirring, to −10° C. Triethylamine (32.2 g. 44.35 ml) was added in one portion followed by the dropwise addition of PCl₃ (6.7 ml) while maintaining the reaction temperature between −15 to −10° C. The reaction mixture was further stirred for 0.5 h at −15 to −10° C. N⁴-Benzoyl-5′-O-(4,4′-dimethoxytrityl)-2′-deoxycytidine (DMT-Bz-C-OH) (12.4 g, from Transgenomic Bioconsumables Ltd) in THF (347 ml) was added to the reaction mixture over a 1 h period and the mixture was then stirred at −10° C. for a further period of 1 h. The reaction mixture was then added to a stirred mixture of triethylamine: H₂O, (1:1, 200 ml) at −10° C. over a period of 15 minutes and allowed to warm to room temperature before being transferred to a separating funnel. The bottom layer was discarded while the top layer was concentrated in vacuo. DCM (580 ml) was added to the residue and the resulting solution was washed with TEAP (0.5 M, 2×75 ml). The reaction mixture was concentrated in vacuo to yield 14.75 g of product (94% yield).

Stage 3

### Synthesis of N⁴-benzoyl-5′-O-(4,4′-dimethoxytrityl)cytidin-3′-yl-N⁴-benzoyl-2′-deoxy-3′-(4-oxopentanoate)-cytidin-5′-yl H-phosphonate (C—C dimer)

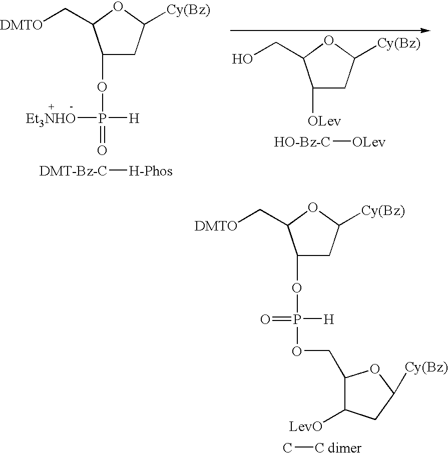

Prior to use all glassware was dried in an oven and cooled in a desiccator. NV-Benzoyl-5′-O-(4,4′-dimethoxytrityl)-2′-deoxy-3′-(hydrogen phosphate)cytidine triethylammonium salt (DMT-Bz-C-H-Phos) (1.1 g, prepared as described in Stage 2) and N⁴-benzoyl-2′-deoxy-3′-(4-oxopentanoate)cytidine (HO-Bz-C-OLev) (0.5 g, from Transgenomic Bioconsumables Ltd) were dried from an azeotropic mixture with CH₃CN (2×25 ml) and toluene (25 ml). The residue was transferred to a 50 ml round-bottomed flask fitted with a nitrogen inlet and dry DMF (10 ml) and dry pyridine (0.56 ml) were added. The mixture was cooled to 0° C. and diphenyl chlorophosphate (0.59 ml in dry DCM (3 ml)) was added dropwise over 2 minutes. The reaction was held at 0° C. for 15 minutes before being quenched by the addition of pH 7 phosphate buffer (5 ml, supplied by Fisher). Saturated aqueous NaHCO₃ (40 ml) was then added to the mixture followed by DCM (40 ml). The lower organic layer was separated and washed with TEAP (0.5 M, 30 ml) and then dried over Na₂SO₄. The title compound (C—C dimer) was stored as a dried DCM solution over Na₂SO₄ in a nitrogen flushed flask at 4° C. to minimise decomposition. Coupling of DMT-Bz-C-H-Phos and HO-Bz-C-OLev to provide the C—C dimer was quantitative by liquid chromatography. However, the C—C dimer, as produced also contained as impurities unreacted pyridine, DMF and (PhO)₂P(O)(OH). Therefore in subsequent experiments the calculated mass of C—C dimer was proportionally increased to compensate for the additional components present within the crude material.

Prior to use the C—C dimer mixture was filtered to remove Na₂SO₄ and concentrated in vacuo.

Stage 4

### Preparation of N,O-bis(tert-butyldimethylsilyl)acetamide (BMTBSA)

Prior to use all glassware was dried in an oven and cooled in a desiccator. Acetamide (7.13 g) was charged to a 1 L round-bottomed flask fitted with a thermometer, nitrogen inlet and overhead stirrer. Dry triethylamine (340 ml, pre-dried over CaH₂) was added and the solution was cooled to 0° C. TBDMSCI (47.37 g) was then added with vigorous stirring. The reaction mixture was vigorously stirred for 22 h and then filtered under nitrogen using dried glassware before being concentrated in vacuo. The resultant crude product mixture was distilled using a Kugelrohr apparatus under 0.6-0.8 mm Hg pressure and at a temperature of from 85 to 100° C. The distilled material solidified to a white solid (14.85 g) which was postulated to be a 2:1 mixture of the di- and mono-silylated acetamide. This was determined from ¹H NMR analysis where the major component was identified as BMTBSA giving signals in agreement with those reported in the literature (J. Org. Chem, 1982, 47, 3336-3339). The minor component contained one TBDMS functional group with ¹H NMR signals consistent with those expected for the mono-silylated acetamide. The mono-silylated acetamide was assumed to be of similar activity to BMTBSA, therefore in subsequent experiments the mass of BMTBSA used was calculated based on the assumption that the crude BMTBSA material was 100% pure.

Stage 5

### Reaction of the C—C dimer with BMTBSA and PADS

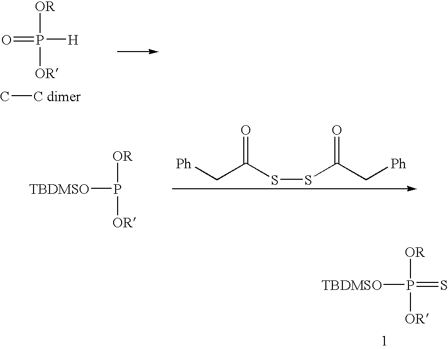

Prior to use all glassware was dried in an oven and cooled in a desiccator. BMTBSA was warmed to melt the solid and was then measured by volume in an air tight syringe which had been heated in the oven immediately prior to use to prevent solidification of the solid (the density of BMTBSA was taken as d=0.859 (J. Org. Chem, 1982, 47, 3336-3339)).

C—C dimer (1.007 g, prepared as described in Example 1, Stage 1 and Stage 2) was charged to a 25 ml round-bottomed flask fitted with nitrogen inlet and dissolved in dry DCM (5 ml). BMTBSA (0.90 ml, 5 equiv, prepared in Example 1, Stage 3) was added to the flask. The reaction mixture was stirred for 5 minutes. PADS (327 mg, 2 equivalents, from Hasegawa Co., Ltd) was then added and the mixture was stirred for a further 5 minutes. During this time the reaction mixture changed from a yellow to a deep purple solution. The reaction mixture was poured onto water (100 ml) and the organic layer was separated. The aqueous layer was further extracted with DCM (3×50 ml). The organic layers were combined and washed with saturated aqueous NaHCO₃ (2×50 ml) and brine (2×50 ml) and dried over Na₂SO₄. Filtration and concentration in vacuo gave 1.82 g of a purple liquid which solidified on standing.

The crude product was analysed by liquid chromatography where the product (1) retention time was 11.1 minutes (17%).

### EXAMPLE 2

### Reaction of the C—C dimer with BMTBSA and 3-amino-1,2,4-dithiazole-5-thione

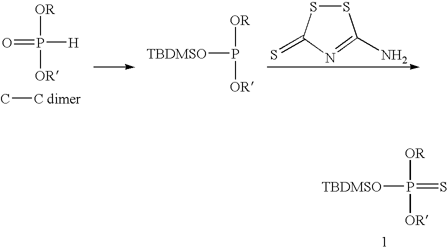

Prior to use all glassware was dried in an oven and cooled in a desiccator. C—C dimer (1.007 g, prepared as described in Example 1, Stage 1 and Stage 2) was charged to a 25 ml round-bottomed flask fitted with a nitrogen inlet and dissolved in dry DCM (5 ml). BMTBSA (0.90 ml, 5 equiv, prepared in Example 1, Stage 3) was then added to the flask and the reaction mixture was stirred for 5 minutes. 3-Amino-1,2,4-dithiazole-5-thione (162 mg, 2 equivalents from Lancaster) was then added and stirring was continued for a further 5 minutes. The reaction mixture was poured onto water (100 ml) and the organic layer was separated. The aqueous layer was further extracted with DCM (3×50 ml). Organic layers were combined and washed with saturated aqueous NaHCO₃ (2×50 ml) and brine (2×50 ml) and dried over Na₂SO₄. Filtration and concentration in vacuo gave 1.10 g of a pale yellow solid.

The crude product was analysed by liquid chromatography and the main product was identified as compound (1) (68% yield) which had a retention time of 10.9 minutes in the liquid chromatography system described above.

1. An oligonucleotide comprising at least one pentavalent internucleotide phosphorus atom protected with a group of formula —X^{a}SiR³R⁴R⁵ wherein X^{a} represents O or S, and R³, R⁴ and R⁵ each independently are optionally substituted hydrocarbyl groups, selected such that that total number of carbon atoms in R³ plus R⁴ plus R⁵ is 4 or more.

2. An oligonucleotide according to claim 1, wherein the group of formula —X^{a}SiR³R⁴R⁵ is a tert-butyldimethylsilyloxy group.

3. An oligonucleotide according to either of claims 1 and 2, wherein a single group of formula —X^{a}SiR³R⁴R⁵ is located at the terminal internucleotide linkage.

4. An oligonucleotide according to claim 1, having the Formula (1): 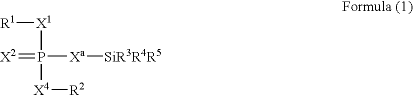
wherein:
R¹ and R² independently are nucleoside, nucleotide or oligonucleotide moieties;
R³, R⁴ and R⁵ each independently are optionally substituted hydrocarbyl groups,
selected such that that total number of carbon atoms in R³ plus R⁴ plus R⁵ is 4 or more;
X^{a} represents O or S;
X¹ and X⁴ are each independently —O—, —CH₂—, —S— or NRⁿ, where Rⁿ represents H or C₁₋₄ alkyl; and X² is O or S.

5. An oligonucleotide according to claim 4, wherein X¹, X^{a} and X⁴ are each O, and one of R³, R⁴ and R⁵ represents a tert-butyl group, with the others representing methyl groups.

6. An oligonucleotide according to either claims 4 and 5, wherein R¹ is a nucleotide substituted at the 3′-position by X¹, and R² represents an oligonucleotide substituted at the 5′-position by X⁴.

7. An oligonucleotide according to claim 4, of Formula (2): 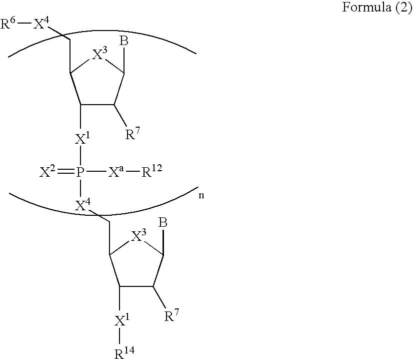
wherein:
X^{a} for each occurrence is independently —O— or S—;
X¹ and X⁴ are, independently, —O—, —CH₂—, —S— or NRⁿ, where Rⁿ represents H or C₁₋₄ alkyl;
X² for each occurrence is O or S;
X³ for each occurrence is, independently, —O—, —S—, —CH₂—, or —(CH₂)₂—;
R⁶ is H, an alcohol protecting group, an amino protecting group or a thio protecting group;
R⁷ for each occurrence is, independently, —H, —F—OR⁸, —NR⁹R¹⁰, —SR¹¹, or a substituted or unsubstituted aliphatic group;
R⁸ for each occurrence is, independently, —H, a substituted or unsubstituted aliphatic group, a substituted or unsubstituted aryl group, a substituted or unsubstituted aralkyl group, an alcohol protecting group, or —(CH₂)_{q}—NR^{x}R^{y};
R⁹ and R¹⁰ for each occurrence are each, independently, —H, a substituted or unsubstituted aliphatic group, or an amine protecting group, or R⁹ and R¹⁰ taken together with the nitrogen to which they are attached are a heterocyclyl group;
R¹¹ for each occurrence is, independently, —H, a substituted or unsubstituted aliphatic group, or a thio protecting group;
R¹² for each occurrence is, independently, a phosphorus protecting group, provided that at least one R¹² represents a group of formula —SiR³R⁴R⁵, in which R³, R⁴ and R⁵ are each independently optionally substituted hydrocarbyl groups, selected such that that total number of carbon atoms in R³ plus R⁴ plus R⁵ is 4 or more;
R¹³ is for each occurrence is, independently, a substituted or unsubstituted aliphatic group, a substituted or unsubstituted aryl group or a substituted or unsubstituted aralkyl group;
R¹⁴ is H a hydroxy protecting group, a thio protecting group, an amino protecting group, —(CH₂)_{q}—NR^{x}R^{y}, a solid support, or a cleavable linker attached to a solid support;
R^{x} and R^{y} are each, independently, —H, a substituted or unsubstituted aryl group, a substituted or unsubstituted heteroaryl group, a substituted or unsubstituted aliphatic group, a substituted or unsubstituted aralkyl group, a substituted or unsubstituted heteroaralkyl group or an amine protecting group, or, R^{x} and R^{y} taken together with the nitrogen to which they are attached form a heterocyclyl group;
q is an integer from 1 to about 6;
B is —H, a natural or unnatural nucleobase, or a protected natural or unnatural nucleobase; and
n is a positive integer.

8. An oligonucleotide according to claim 7, wherein each X¹, X³ and X⁴ are O; R⁶ is H or an alcohol protecting group; R⁷ is H, F, OCH₃, OCH₂CH₂OCH₃ or O-protecting group; R¹² is —CH₂CH₂CN or tert-butyldimethylsilyl, provided at least one R¹² is tert-butyidimethylsilyl; R¹⁴ is H or a cleavable linker attached to a solid support, and n is from 8 to 40.

9. A process for the preparation of a compound of Formula (1) as defined in claim 4, which comprises oxidising or sulfurising a compound of Formula (3): 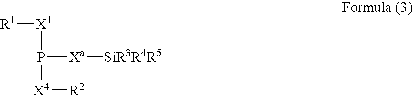
wherein R¹, R², R³, R⁴, R⁵, X^{a}, X¹ and X⁴ are as defined in claim 4.

10. (canceled)

11. A compound of Formula (4):
R¹—X¹—P(NR¹⁷R¹⁸)—X^{a}—SiR³R⁴R⁵
wherein,
R¹ is a nucleoside, nucleotide or oligonucleotide moiety;
R³, R⁴ and R⁵ each independently are optionally substituted hydrocarbyl groups,
selected such that that total number of carbon atoms in R³ plus R⁴ plus R⁵ is 4 or more;
X^{a} represents O or S;
X¹ is —O—, —CH₂—, —S— or NRⁿ, where Rⁿ represents H or C₁₋₄ alkyl; and
R¹⁷ and R¹⁸ are each, independently, a substituted or unsubstituted aliphatic group, a substituted or unsubstituted aryl group, a substituted or unsubstituted aralkyl or R¹⁷ and R¹⁸ taken together with the nitrogen to which they are bound form a heterocyclyl group.

12. A process for the preparation of a compound of Formula (1) 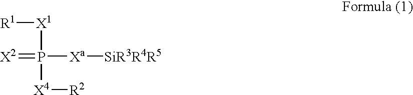
which comprises:
a) coupling a compound of Formula (4),
R¹—X¹—P(NR¹⁷R¹⁸)—X^{a}—SiR³R⁴R⁵
with a compound of formula R²—X⁴—H, in the presence of an activator; and b) oxidising or sulfurising the product of step a) wherein
R¹ and R² independently are nucleoside, nucleotide or oligonucleotide moieties;
R³, R⁴ and R⁵ each independently are optionally substituted hydrocarbyl groups, selected such that that total number of carbon atoms in R³ plus R⁴ plus R⁵ is 4 or more;
X^{a} represents O or S;
X¹ and X⁴ are each independently —O—, —CH₂—, —S— or NRⁿ, where Rⁿ represents H or C₁₋₄ alkyl;
X² is O or S; and
R¹⁷ and R¹⁸ are each, independently, a substituted or unsubstituted aliphatic group, a substituted or unsubstituted aryl group, a substituted or unsubstituted aralkyl or R¹⁷ and R¹⁸ taken together with the nitrogen to which they are bound form a heterocyclyl group.

13. A process for the preparation of a compound of Formula (3) 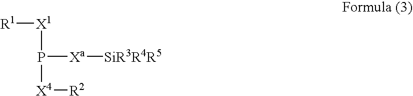
which comprises coupling a compound of Formula (4)
R¹—X¹—P(NR¹⁷R¹⁸)—X^{a}—SiR³R⁴R⁵
with a compound of formula R²—X⁴—H, in the presence of an activator
wherein
R¹ and R² independently are nucleoside, nucleotide or oligonucleotide moieties;
R³, R⁴ and R⁵ each independently are optionally substituted hydrocarbyl groups, selected such that that total number of carbon atoms in R³ plus R⁴ plus R⁵ is 4 or more;
X^{a} represents O or S;
X¹ and X⁴ are each independently —O—, —CH₂—, —S— or NRⁿ, where Rⁿ represents H or C₁₋₄ alkyl; and
R¹⁷ and R¹⁸ are each, independently, a substituted or unsubstituted aliphatic group, a substituted or unsubstituted aryl group, a substituted or unsubstituted aralkyl or R¹⁷ and R¹⁸ taken together with the nitrogen to which they are bound form a heterocyclyl group.

14. A process for the preparation of a compound of Formula (4)
R¹—X¹—P(NR¹⁷R¹⁸)—X^{a}—SiR³R⁴R⁵
which comprises reacting a compound of formula R¹—X¹—H,
with a compound of formula R³R⁴R⁵Si—X^{a}—P(NR¹⁷R¹⁸)₂ wherein
R¹ is a nucleoside, nucleotide or oligonucleotide moiety;
R³, R⁴ and R⁵ each independently are optionally substituted hydrocarbyl groups, selected such that that total number of carbon atoms in R³ plus R⁴ plus R⁵ is 4 or more:
X^{a} represents O or S:
X¹ is —O—, —CH₂—, —S— or NRⁿ, where Rⁿ represents H or C₁₋₄alkyl; and
R¹⁷ and R¹⁸ are each, independently, a substituted or unsubstituted aliphatic group, a substituted or unsubstituted aryl group, a substituted or unsubstituted aralkyl or R¹⁷ and R¹⁸ taken together with the nitrogen to which they are bound form a heterocyclyl group.

15. A process for the preparation of a compound of Formula (4)
R¹—X¹—P(NR¹⁷R¹⁸)—X^{a}—SiR³R⁴R⁵
wherein X^{a} is 0 which comprises a) reacting a compound of formula R¹—X¹—H, with a compound of formula Z-P(NR¹⁷R¹⁸)₂ to form a compound of formula R¹—X¹—P(NR¹⁷R¹⁸)₂; b) hydrolysing the compound of formula R¹—X¹—P(NR¹⁷R¹⁸)₂ to form a compound of formula R¹—X¹—PH(═O)(NR¹⁷R¹⁸), and c) reacting the compound of formula R¹—X¹—PH(═O)(NR¹⁷R¹⁸) with a silylating agent of formula Y¹—SiR³R⁴R⁵ wherein Y¹ is a leaving group, to form the compound of Formula (4)
wherein
R¹ is a nucleoside, nucleotide or oligonucleotide moiety;
R³, R⁴ and R⁵ each independently are optionally substituted hydrocarbyl groups, selected such that that total number of carbon atoms in R³ plus R⁴ plus R⁵ is 4 or more;
X¹ and X⁴ are each independently —O—, —CH₂—, —S— or NRⁿ, where Rⁿ represents H or C₁₋₄ alkyl:
R¹⁷ and R¹⁸ are each, independently, a substituted or unsubstituted aliphatic group, a substituted or unsubstituted aryl group, a substituted or unsubstituted aralkyl or R¹⁷ and R¹⁸ taken together with the nitrogen to which they are bound form a heterocyclyl group;
Y¹ and Z each independently represent a leaving group.

16. (canceled)

17. (canceled)

18. (canceled)

19. (canceled)

20. (canceled)

21. (canceled)

22. (canceled)

23. (canceled)

24. An oligonucleotide according to claim 4 wherein X¹, X^{a} and X⁴ are each O and X² is S.
